(19) United States
(12) Patent Application Publication
(10) Pub. No.: US 2026/0229138 A1
(43) Pub. Date: Aug. 6, 2026

(54) INTEGRATED ROBOTIC SURGICAL SYSTEM SIMULATOR AND METHOD FOR PERFORMING PSYCHOMOTOR MEMORY EXERCISES

(71) Applicant: AURIS HEALTH, INC., Santa Clara, CA (US)

(72) Inventors: Julien LANG, Santa Clara, CA (US); Elizabeth FINN, Santa Clara, CA (US); Emma ESSOCK-BURNS, Santa Clara, CA (US)

(21) Appl. No.: 19/656,717

(22) Filed: Apr. 23, 2026

Related U.S. Application Data
(63) Continuation of application No. PCT/IB2024/060609, filed on Oct. 28, 2024.
(60) Provisional application No. 63/595,045, filed on Nov. 1, 2023.
(60) Provisional application No. 63/595,050, filed on Nov. 1, 2023.

(51) Int. Cl. G09B 9/00; A61B 34/10; A61B 34/35

(52) U.S. Cl. CPC G09B 9/00; A61B 34/35; A61B 2034/101

(57) ABSTRACT

An integrated robotic surgical system simulator and method for providing psychomotor memory exercises are provided. In one embodiment, a robotic surgical system user console is provided including a plurality of user input devices; a simulator configured to provide a simulation exercise; and a switch. When in clinical mode, the switch causes signals generated in response to user-manipulation of the plurality of user input devices to cause movement of robotic arms of the robotic surgical system. However, when in simulation mode, the switch causes signals generated in response to user-manipulation of the plurality of user input devices to be provided to the simulator for the simulation exercise but do not cause movement of robotic arms of the robotic surgical system. Other embodiments are provided.

### CROSS-REFERENCE TO RELATED APPLICATIONS

The present patent document is a bypass continuation and claims priority to PCT/IB2024/060609, filed Oct. 28, 2024, (Docket No. 010336-20023C-WO), which claims the benefit of the filing date under 35 U.S.C. § 119(e) of Provisional U.S. Patent Application Serial Nos. 63/595,045, filed Nov. 1, 2023, and 63/595,050, filed Nov. 1, 2023, all of which are hereby incorporated by reference.

### TECHNICAL FIELD

The following embodiments generally relate to the field of robotic surgery and more specifically to a robotic surgical system simulator.

### BACKGROUND

Minimally-invasive surgery (MIS), such as laparoscopic surgery, involves techniques intended to reduce tissue damage during a surgical procedure. For example, laparoscopic procedures typically involve creating a number of small incisions in the patient (e.g., in the abdomen), and introducing one or more surgical instruments (e.g., an end effector, at least one camera, etc.) through the incisions into the patient. The surgical procedures may then be performed using the introduced surgical instruments, with the visualization aid provided by the camera.

Generally, MIS provides multiple benefits, such as reduced patient scarring, less patient pain, shorter patient recovery periods, and lower medical treatment costs associated with patient recovery. In some embodiments, MIS may be performed with robotic systems that include one or more robotic arms for manipulating surgical instruments based on commands from an operator. A robotic arm may, for example, support at its distal end various devices, such as surgical end effectors, imaging devices, cannulae for providing access to the patient's body cavity and organs, etc.

Simulators have been used to assist surgeons in becoming familiar with using a robotic surgical system.

### BRIEF DESCRIPTION OF THE DRAWINGS

FIG. 1A depicts an example of an operating room arrangement with a robotic surgical system and a user console of an embodiment.

FIG. 1B is a schematic illustration of one exemplary variation of a robotic arm manipulator, tool driver, and cannula with a surgical tool of an embodiment.

FIG. 1C is a schematic illustration of an exemplary user console of an embodiment.

FIG. 2 is a schematic illustration of an exemplary variation of a user console for a robotic surgical system of an embodiment in communication with one or more third party devices.

FIG. 3 is a schematic of a surgical robotic platform of an embodiment with a graphical user interface (GUI) module, where the surgical robotic platform is in communication with multiple medical data resources.

FIGS. 4A and 4B are perspective and longitudinal cross-sectional views, respectively, of one exemplary variation of a handheld user input device of an embodiment.

FIGS. 5A and 5B are schematic illustrations of an exemplary variation of a robotic surgical system with an integrated simulator.

FIG. 6 is a flow chart of a method of an embodiment for providing a training exercise on a simulator.

FIG. 7 is an illustration of an embodiment that shows that a given position of a user input device (UID) determines a workspace of a tool.

FIG. 8 is an illustration of an embodiment that shows that, in active teleoperation, moving the UID moves the tool without moving the workspace.

FIG. 9 is an illustration of an embodiment that shows that, when clutching, moving the UID moves the tool workspace in the opposite direction.

FIG. 10 is an illustration of an embodiment that shows that the UID workspace to tool workspace is independent for each tool.

FIG. 11 is an illustration of an embodiment that shows that moving a camera clutches the instrument.

### DETAILED DESCRIPTION

Non-limiting examples of various aspects and variations of the embodiments are described herein and illustrated in the accompanying drawings.

### Robotic Surgical System Overview

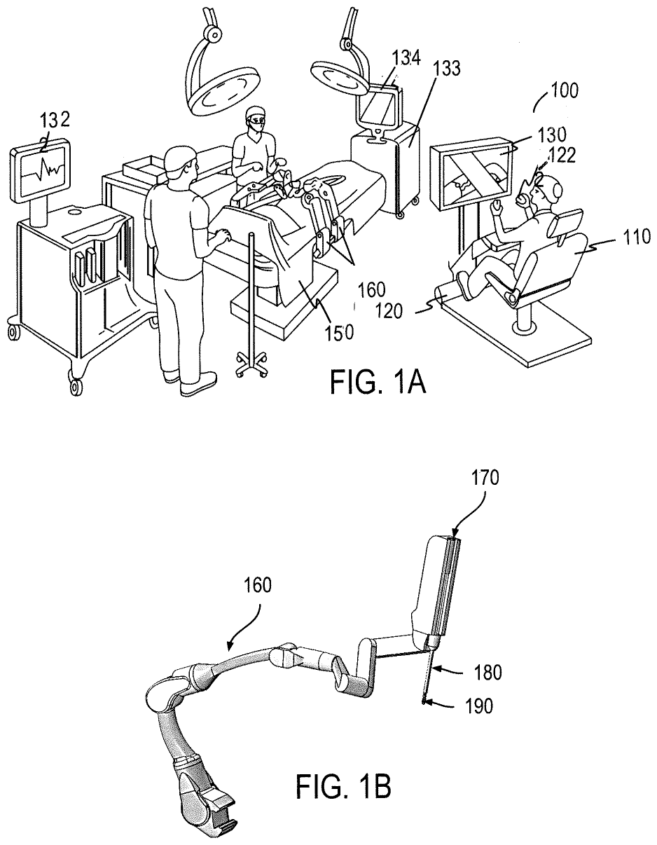

FIG. 1A is an illustration of an exemplary operating room environment with a robotic surgical system. Generally, as shown in FIG. 1A, the robotic surgical system includes a user console 100 (sometimes referred to herein as the “surgeon bridge” or “bridge”), a control tower 133, and one or more robotic arms 160 located at a robotic platform (e.g., table, bed, etc.), where surgical instruments (e.g., with end effectors) are attached to the distal ends of the robotic arms 160 for executing a surgical procedure. The robotic arms 160 are shown as a table-mounted system, but in other configurations, one or more robotic arms may be mounted to a cart, ceiling or sidewall, or other suitable support surface.

As further illustration, as shown in the exemplary schematic of FIG. 1B, a robotic surgical system may include at least one robotic arm 160 and a tool driver 170 generally attached to a distal end of the robotic arm 160. A cannula 180 coupled to the end of the tool driver 170 may receive and guide a surgical instrument 190 (e.g., end effector, camera, etc.). Furthermore, the robotic arm 160 may include a plurality of links that are actuated so as to position and orient the tool driver 170, which actuates the surgical instrument 190.

Generally, as shown in FIG. 1A, the user console 100 may be used to interface with the robotic surgical system 150. A user (such as a surgeon or other operator) may use the user console 100 to remotely manipulate the robotic arms 160 and/or surgical instruments (e.g., in tele-operation). The user console 100 may be located in the same operating room as the robotic system 150, as shown in FIG. 1A. In other embodiments, the user console 100 may be located in an adjacent or nearby room, or tele-operated from a remote location in a different building, city, or country. In one example, the user console 100 may comprise a seat 110, foot-operated controls (pedals) 120, one or more handheld user input devices 122, and at least one user display 130 configured to display, for example, a view of the surgical site inside a patient (e.g., captured with an endoscopic camera), and/or other surgical or medical information.

In the exemplary user console shown in FIG. 1C, a user located in the seat 110 and viewing the user display 130 may manipulate the foot-operated controls 120 and/or handheld user input devices 122 to remotely control the robotic arms 160 and/or surgical instruments mounted to the distal ends of the arm. The foot-operated controls 120 and/or handheld user input devices 122 may additionally or alternatively be used to control other aspects of the user console 100 or robotic system 150. For example, in variations in which the user generally controls (at any given time) a designated “left-hand” robotic arm/instrument and a designated “right-hand” robotic arm/instrument, the foot-operated controls 120 may enable a user to designate from among a larger group of available robotic arms/instruments which robotic arms/instruments comprise the “left-hand” and “right-hand” robotic arm/instruments (e.g., via toggle or rotation in selection among the available robotic arms/instruments). Other examples include adjusting or configuring the seat 110, the foot-operated controls 120, the user input devices 122, and/or the user display 130.

In some variations, a user may operate the surgical robotic system in an “over the bed” (OTB) mode, in which the user is at the patient's side and simultaneously manipulating a robotically-driven instrument/end effector attached thereto (e.g., with a handheld user input device 122 held in one hand) and a manual laparoscopic tool. For example, the user's left hand may be manipulating a handheld user input device 122 to control a robotic surgical component, while the user's right hand may be manipulating a manual laparoscopic tool. Accordingly, in these variations, the user may perform both robotic-assisted MIS and manual laparoscopic surgery on a patient.

During an exemplary procedure or surgery, the patient is prepped and draped in a sterile fashion, and anesthesia may be achieved. Initial access to the surgical site may be performed manually with the robotic system 150 in a stowed configuration or withdrawn configuration to facilitate access to the surgical site. Once access is completed, initial positioning and/or preparation of the robotic system may be performed. During the surgical procedure, a surgeon or other user in the user console 100 may utilize the foot-operated controls 120, user input devices 122, and/or other suitable controls to manipulate various end effectors and/or imaging systems to perform the procedure. Manual assistance may be provided at the procedure table by other personnel, who may perform tasks including but not limited to retracting tissues, or performing manual repositioning or tool exchange involving one or more robotic arms 160. Other personnel may be present to assist the user at the user console 100. Medical and surgery-related information to aid other medical personnel (e.g., nurses) may be provided on additional displays such as a display 134 on a control tower 133 (e.g., control system for the robotic surgical system) and/or a display 132 located bedside proximate the patient. For example, as described in further detail herein, some or all information displayed to the user in the user console 100 may also be displayed on at least one additional display for other personnel and/or provide additional pathways for inter-personnel communication. When the procedure or surgery is completed, the robotic system 150 and/or user console 100 may be configured or set in a state to facilitate one or more post-operative procedures, including but not limited to robotic system cleaning and/or sterilization, and/or healthcare record entry or printout, whether electronic or hard copy, such as via the user console 100.

In some variations, the communication between the robotic system 150, the user console 100, and any other displays may be through the control tower 133, which may translate user commands from the user console 100 to robotic control commands and transmit them to the robotic system 150. The control tower 133 may transmit status and feedback from the robotic system 150 back to the user console 100 (and/or other displays). The connections between the robotic system 150, the user console 100, other displays, and the control tower 133 may be via wired and/or wireless connections, and may be proprietary or performed using any of a variety of data communication protocols. Any wired connections may be built into the floor and/or walls or ceiling of the operating room. The robotic surgical system may provide video output to one or more displays, including displays within the operating room as well as remote displays accessible via the Internet or other networks. The video output or feed may be encrypted to ensure privacy, and all or one or more portions of the video output may be saved to a server, an electronic healthcare record system, or other suitable storage medium.

In some variations, additional user consoles 100 may be provided, for example to control additional surgical instruments, and/or to take control of one or more surgical instruments at a primary user console. This will permit, for example, a surgeon to take over or illustrate a technique during a surgical procedure with medical students and physicians-in-training, or to assist during complex surgeries requiring multiple surgeons acting simultaneously or in a coordinated manner.

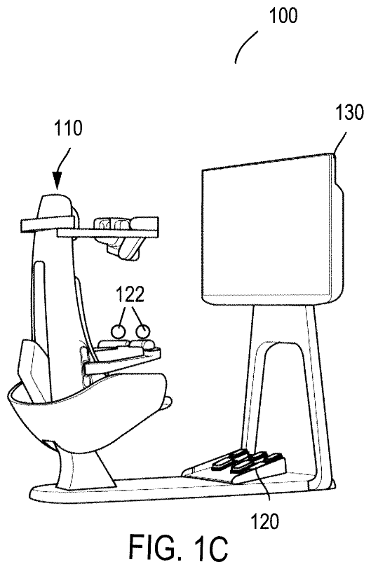

In some variations, as shown in the schematic illustration of FIG. 2, one or more third party devices 240 may be configured to communicate with the user console 210 and/or other suitable portions of the robotic surgical system. For example, as described elsewhere herein, a surgeon or other user may sit in the user console 210, which may communicate with the control tower 230 and/or robotic instruments in a robotic system 220. Medical data (e.g., endoscopic images, patient vitals, tool status, etc.) may be displayed at the user console 210, the control tower 230, and/or other displays. At least a subset of the surgical and other medical-related information may furthermore be displayed at a third party device 240, such as a remote computer display that is viewed by a surgical collaborator in the same room or outside the room. Other communication, such as teleconferencing with audio and/or visual communication, may further be provided to and from the third party device. The surgical collaborator may be, for example, a supervisor or trainer, a medical colleague (e.g., radiologist), or other third party who may, for example, view and communicate via the third party device 240 to assist with the surgical procedure.

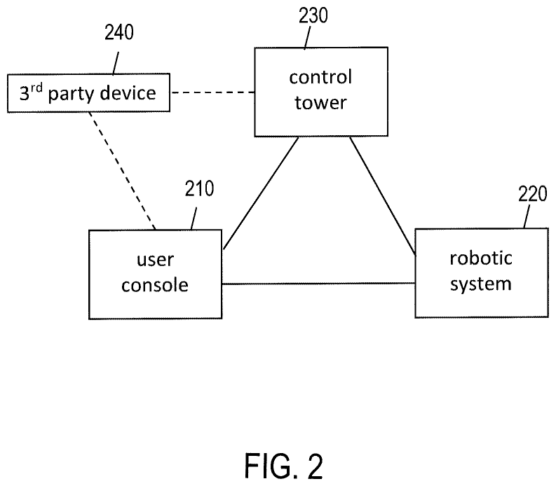

FIG. 3 is a schematic illustration of an exemplary variation of a system 300 including a robotic surgical system and its interaction with other devices and parties. Although a particular architecture of the various connected and communicating systems is depicted in FIG. 3, it should be understood that in other variations, other suitable architectures may be used and the arrangement shown in FIG. 3 is for illustrative purposes. The system 300 may include a surgical robotic platform 302 that facilitates the integration of medical data from discrete medical data resources generated from a variety of parties. Data from the discrete medical data resources may, for example, be used to form temporally coordinated medical data. Multi-panel displays of the temporally coordinated medical data may be configured and presented, as described further herein.

The platform 302 may be, for example, a machine with one or more processors 310 connected to one or more input/output devices 312 via a bus 314. The at least one processor may, for example, include a central processing unit, a graphics processing unit, an application specific integrated circuit, a field programmable logic device or combinations thereof.

The surgical robotic platform 302 may include one or more input ports to receive medical data from discrete medical data resources. For example, a surgical robot port 329 may receive surgical robot data from a surgical robot 330. Such data may, for example, include position data or other suitable status information. An imaging port 331 may receive imaging data from an imaging device 332, such as an endoscope, that is configured to capture images (e.g., still images, video images) of a surgical site. The endoscope may, for example, be inserted through a natural orifice or through an aperture in a surgical patient. As another example, one or more medical instrumentation ports 333 may receive patient vital information from medical instrumentation 334 (e.g., a pulse oximeter, electrocardiogram device, ultrasound device and/or the like). Additionally, as another example, one or more user control data ports 335 may receive user interaction data from one or more control devices that receive user inputs from a user for controlling the system. For example, one or more handheld user input devices, one or more foot pedals, and/or other suitable devices (e.g., eye tracking, head tracking sensors) may receive user inputs.

The surgical robotic platform 302 may further include one or more output ports 337 configured for connection to one or more displays 338. For example, the displays 338 may include an open display (e.g., monitor screen) in a user console, an immersive display or head-mounted device with a display, on supplemental displays such as on a control tower display (e.g., team display), a bedside display (e.g., nurse display), an overhead “stadium” style screen, etc. For example, the graphical user interface disclosed herein may be presented on one or more displays 338. The one or more displays 338 may present three-dimensional images. In some variations, the one or more displays 338 may include a touchscreen. The one or more displays 138 may be a single display with multiple panels, with each panel presenting different content. Alternatively, the one or more displays 138 may include a collection of individual displays, where each individual display presents at least one panel.

In some variations, a network interface 316 may also be connected to the bus 314. The network interface 316 may, for example, provide connectivity to a network 317, which may be any combination of one or more wired and/or wireless networks. The network 317 may, for example, help enable communication between the surgical robotic platform 302 and other data sources or other devices. For example, one or more third party data sources 340 may also be connected to the network 317. The third party source 340 may include a third party device (e.g., another computer operated by a third party such as another doctor or medical specialist), a repository of video surgical procedure data (e.g., which may be relevant to a procedure being performed by a surgeon), or other suitable source of additional information related to a surgical procedure. For example, the third party device data may be ported to a panel that is displayed to a surgeon before, during or after a procedure.

As another example, one or more application databases 342 may be connected to the network 317 (or alternatively, stored locally within a memory 320 within the surgical robotic platform 302). The application database 342 may include software applications (e.g., as described in further detail below) that may be of interest to a surgeon during a procedure. For example, a software application may provide access to stored medical records of a patient, provide a checklist of surgical tasks for a surgical procedure, perform machine vision techniques for assisting with a procedure, perform machine learning tasks to improve surgical tasks, etc. Any suitable number of applications may be invoked. Information associated with an application may be displayed in a multi-panel display or other suitable display during a procedure. Additionally or alternatively, information provided by one or more applications may be provided by separate resources (e.g., a machine learning resource) otherwise suitably in communication with the surgical robotic platform 302.

In some variations, one or more of the software applications may run as a separate process that uses an application program interface (API) to draw objects and/or images on the display. APIs of different complexities may be used. For example, a simple API may include a few templates with fixed widget sizes and locations, which can be used by the GUI module to customize text and/or images. As another example, a more complex API may allow a software application to create, place, and delete different widgets, such as labels, lists, buttons, and images.

Additionally or alternatively, one or more software applications may render themselves for display. This may, for example, allow for a high level of customization and complex behavior for an application. For example, this approach may be implemented by allowing an application to pass frames that are rendered by a graphical user interface (GUI) module 324, which can be computer-readable program code that is executed by the processor 310. Alternatively, an image buffer may be used as a repository to which an application renders itself.

In some variations, one or more software applications may run and render themselves independent of the GUI module 324. The GUI module may still, however, launch such applications, instruct the application or the operating system where the application is to be positioned on the display, etc.

As another approach, in some variations, one or more applications may run completely separate from the GUI rendered by the GUI module. For example, such applications may have a physical video connection and data connection to the system (e.g., through suitable input/output devices, network, etc.). The data connection may be used to configure video feed for an application to be the appropriate pixel dimensions (e.g., full screen, half screen, etc.).

As shown in FIG. 3, in some variations, a memory 320 may also be connected to the bus 314. The memory 320 may be configured to store data processed in accordance with embodiments of the methods and systems described herein.

In some variations, the memory 320 may be configured to store other kinds of data and/or software modules for execution. For example, a user console may include a memory 320 that stores a GUI module 324 with executable instructions to implement operations disclosed herein. The GUI module may, for example, combine and aggregate information from various software applications and/or other medical data resources for display. In some exemplary variations, one or more software applications may be incorporated into base code of the GUI module, such that the module draws graphics and displays text in the appropriate location on the display. For example, the module may fetch the images from a database, or the images may be pushed to the interface from an instrument (e.g., endoscopic camera) in the operating room, via a wired or wireless interface.

In some variations, medical data may be collected from discrete medical data resources (e.g., surgical robot 330, endoscope 332, medical instrumentation 334, control devices 336, third party data source 340, application database 342, etc.). Additionally, at least some of the medical data may be temporally coordinated such that, when necessary, time sensitive information from different medical data resources is aligned on a common time axis. For example, surgical robot position data may be time coordinated with endoscope data, which is coordinated with operator interaction data from control devices. Similarly, a networked resource, such as information provided by one or more software applications, may be presented at an appropriate point in time along with the other temporally coordinated data. Multi-panel displays, and/or other suitable displays, may be configured to communicate medical information (e.g., including the temporally coordinated medical data) as part of a graphical user interface (GUI).

Various exemplary aspects of a GUI for a robotic surgical system are described herein. In some variations, the GUI may be displayed in a multi-panel display at a user console that controls the robotic surgical system. Additionally or alternatively, the GUI may be displayed at one or more additional displays, such as at a control tower for the robotic surgical system, at a patient bedside, etc. Generally, the GUI may provide for more effective communication of information to a user in the user console and/or other personnel, as well as for more effective communication and collaboration among different parties involved in a surgical procedure, as further described below.

### Graphical User Interface (GUI) Interaction

In one embodiment, the GUI is displayed on a display 130 in a user console 100 that is used to control the robotic surgical system 150 (e.g., by a surgeon), and at least some of interactive graphical objects displayed on the display 130 may be controlled, selected, or otherwise interacted with via one or more user controls that are also used to control an aspect of the surgical system (e.g., surgical instrument). For example, a user may use one or more handheld user input devices 122 and/or one or more foot pedals 120 to selectively control an aspect of the robotic surgical system 150 and selectively interact with the GUI. By enabling control of both the robotic surgical system 150 and the GUI with the same user controls, the user may advantageously avoid having to switch between two different kinds of user controls. Enabling the user to use the same input devices to control the robotic system 150 and the GUI streamlines the surgical procedure and increases efficiency, as well as helps the user maintain sterility throughout a surgical procedure.

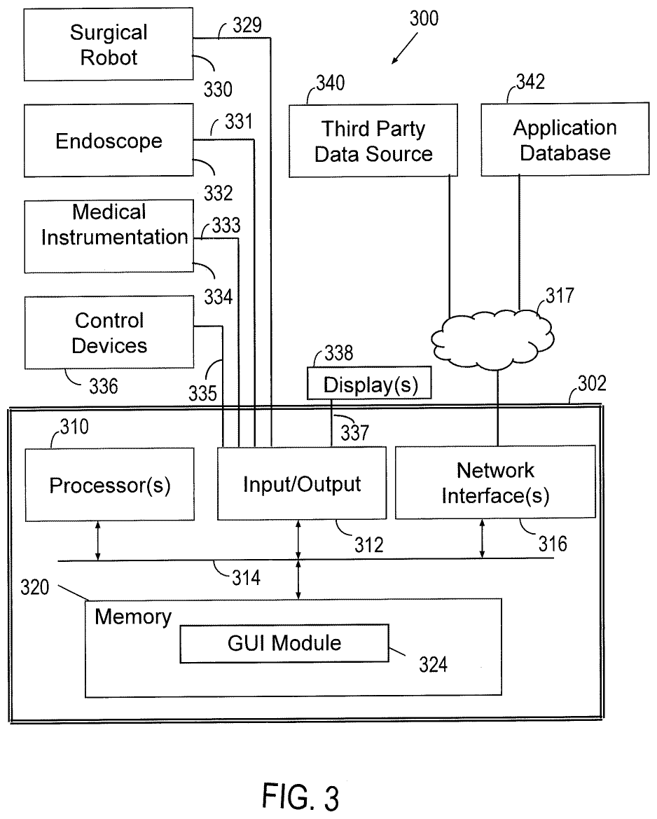

As shown generally in FIGS. 4A and 4B, an exemplary variation of a handheld user input device 122 for controlling a robotic system may include a member 410, a housing 420 at least partially disposed around the member 410 and configured to be held in the hand of a user, and a tracking sensor system 440 configured to detect at least position and/or orientation of at least a portion of the device. The housing 420 may be flexible (e.g., made of silicone). In some instances, the detected position and/or orientation of the device may be correlatable to a control of the robotic system. For example, the user input device 122 may control at least a portion of a robotic arm, an end effector or tool (e.g., graspers or jaws) coupled to a distal end of the robotic arm, a GUI, or other suitable aspect or feature of the robotic surgical system 150. Additionally, in some instances, the detected position and/or orientation of the device 122 may be correlatable to a control of a GUI. Furthermore, in some variations, the user input device 122 may include one or more sensors for detecting other manipulations of the user input device 122, such as squeezing of the housing 420 (e.g., via one or more pressure sensors, one or more capacitive sensors, etc.).

Generally, a user interface for controlling a robotic surgical system may include at least one handheld user input device 122, or may include at least two handheld user input devices 122 (e.g., a first user input device to be held by a left hand of the user, and a second user input device to be held by a right hand of the user), or any suitable number. Each user input device 122 may be configured to control one or more different aspects or features of the robotic system. For example, a user input device held in the left hand of the user may be configured to control an end effector represented on a left side of a camera view provided to the user, while a user input device held in the right hand of the user may be configured to control an end effector represented on a right side of the camera view.

In some variations, the handheld user input device 122 may be a groundless user input device configured to be held in the hand and manipulated in free space. For example, the user input device 122 may be configured to be held between the fingers of a user, and moved about freely (e.g., translated, rotated, tilted, etc.) by the user as the user moves his or her arms, hands, and/or fingers. Additionally or alternatively, the handheld user input device 122 may be a body-grounded user input device, in that the user input device 122 may be coupled to a portion of the user (e.g., to fingers, hand, and/or arms of a user) directly or via any suitable mechanism such as a glove, hand strap, sleeve, etc. Such a body-grounded user input device may still enable the user to manipulate the user input device in free space. Accordingly, in variations in which the user input device 122 is groundless or body-grounded (as opposed to permanently mounted or grounded to a fixed console or the like), the user input device 122 may be ergonomic and provide dexterous control, such as by enabling the user to control the user input device with natural body movements unencumbered by the fixed nature of a grounded system.

The handheld user input device 122 may include wired connections that, for example, may provide power to the user input device 122, carry sensor signals (e.g., from the tracking sensor assembly and/or other sensors such as a capacitive sensor, optical sensor, etc. Alternatively, the user input device may be wireless as shown in FIG. 4A and communicate commands and other signals via wireless communication such as radiofrequency signals (e.g., WiFi or short-range such as 400-500mm range, etc.) or other suitable wireless communication protocol such as Bluetooth. Other wireless connections may be facilitated with optical reader sensors and/or cameras configured to detect optical markers on the user input device 122 infrared sensors, ultrasound sensors, or other suitable sensors.

The handheld user input device may include a clutch mechanism for switching between controlling a robotic arm or end effector and controlling a graphical user interface, etc., and/or between other control modes. One or more of the various user inputs described in further detail below may, in any suitable combination, function as a clutch. For example, touching a gesture touch region of the device, squeezing the housing, flicking or rotating the user input device, etc. may function to engage a clutch. As another example, a combination of squeezing and holding the user input device, and rotating the user input device, may function as a clutch. However, any suitable combination of gestures may function as a clutch. Additionally or alternatively, user input to other user input devices (e.g., foot pedal assembly) may, alone or in combination with user input to a handheld user input device, function as a clutch.

In some variations, engagement and disengagement of a clutch mechanism may enable transition between use of a handheld user input device as a control for the robotic system and use of the handheld user input device as a control for the GUI (e.g., to operate a cursor displayed on the screen). When a clutch mechanism is engaged such that the user input devices are used to control the GUI, positions or poses of the robotic arms may be substantially locked in place to “pause” operation of the robotic system, such that subsequent movement of the user input devices while the clutch is engaged will not inadvertently cause movement of the robotic arms. Simulator

Another variation of an application for a GUI is a simulator application. Surgeons and operating room staff need to be trained under a realistic simulation to acquire skills for usage and successful adoption of the robotic surgical system 150. A simulator application may, for example, be in communication with a database storing simulated surgical robotic experiences or simulated exercises, such as to teach user-specific psychomotor skills for robotic simulation (e.g., games to practice performing a roll action of a handheld user input device and/or other skills, etc.). Running the simulator on the surgeon bridge 100 provides the most-realistic and full-featured training, as the surgeon is using the same UIDs and GUIs as he would during real surgery. Simulated surgical robotic experiences may include, for example, simulation and training exercises with simulated patients. The simulator application may load such simulated experiences into the GUI, including a simulated endoscopic view and other patient parameters. The simulator allows surgeons to build psychomotor skills required for the surgical procedures and provides performance metrics on the simulation. For example, surgeons may be trained by the simulator to manipulate instruments for pick and place, camera targeting, needle driving, suturing, and knot tying. The simulated experiences may further include simulated events, such as robotic arm collisions, patient distress, and other suitable events that may help enable a new user (e.g., a surgeon in training) to learn how to respond and resolve issues appropriately. Simulations may be generated separately from the simulator application, such as with simulation developer software, or alternatively may be generated within the simulator application itself.

In some variations, the simulator application may grade a user based on his or her performance in the simulated exercise, such as by providing a score for the user. Such scores may be tracked over time to gauge a trainee's progress and fluency in using the robotic surgical system. In some variations, the simulator application may display a user's progress throughout a set curriculum (e.g., indicating a user has completed three out of ten exercises), evaluate baseline skills of the user to tailor or adjust curriculum, and/or provide recommendations for particular simulation exercises based on the user's performance.

In one embodiment, the hardware/software components of the simulator are integrated into the user console/surgeon bridge 100 of the robotic surgical system 150 (e.g., in the same housing as other components of the console/surgeon bridge 100). Prior simulators are separate components that are removably attached to the external surface of the user console, with data cables plugged into the user console and a power cable plugged into a power outlet. Thus, setting up this external component can take several minutes before it is ready to use, and additional time is required to tear-down the components after use. Further, the extra cables can create a tripping or other hazard. In contrast to these master-slave training stations, with the simulator integrated in the surgeon bridge 100, a user simply clicks a button on the GUI for the simulator application, and the simulator is ready for use (perhaps after a short reset time) without requiring the user to worry about plugging in data and power cables and waiting for other components to boot.

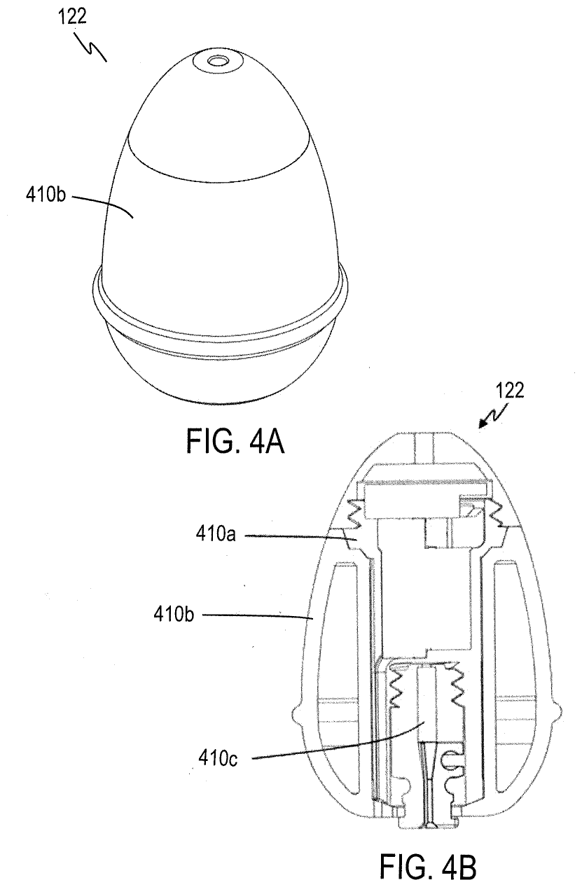

Returning to the drawings, FIGS. 5A and 5B are illustrations of a surgeon bridge 100 with an integrated simulator of an embodiment. As shown in these figures, in this embodiment, the surgeon bridge 100 comprises a surgeon bridge mode switch 510, a surgeon bridge computer 520 (which can be a first processor), a simulator control computer 540 (which can be a second processor), and simulator rendering computer 550 (which can be a third processor). The surgeon bridge mode switch 510 can be placed in communication with a control tower switch 530 (which provides communication to a robotic arm controller), and the simulator control computer 540 can be placed in communication with a network cloud 580 (e.g., the Internet).

The surgeon bridge computer 520 accepts signals from the surgeon bridge user input devices (e.g., UIDs, pedals, and displays) and provides them to the surgeon bridge mode switch 510. In an actual surgical procedure, these signals are provided to the control tower switch 530, which uses the signals to manipulate the surgical robot. The control tower switch 530 can also provide feedback and other signals to the surgeon bridge computer 520 for display on the GUI of the surgeon bridge 100.

In this embodiment, the surgeon bridge computer 520 and the simulator control computer 540 form the simulator. In operation, the simulator control computer 540 provides the hardware/software to create the simulation, accepting and reacting to user input from the surgeon bridge computer 520 that is normally used to control the robot. The simulator control computer 540 can be connected to the cloud 580 (e.g., the Internet) to download simulation software and upload simulation results, for example. The simulator rendering computer 550 communicates with the simulator control computer 540 to provide a visual rendering of the simulation on the GUI displayed on the display of the surgeon bridge 100.

In this embodiment, the surgeon bridge computer 520 and the simulator control computer 540 are integrated in the surgeon bridge 100 in that those components are in the same housing as the surgeon bridge computer 520 and other components (some of which not shown in the drawings) of the surgeon bridge 100. In one embodiment, the simulator control computer 540 is a separate computer system with independent processors, memory, and storage from the surgeon bridge computer 520. In an alternate embodiment, the simulator control computer 540 and the surgeon bridge computer 520 share some components. In yet another alternate embodiment, the simulator is run as a virtual machine on the surgeon bridge computer 520.

It should be noted that while the simulator in this embodiment comprises the surgeon control computer 540 and the simulator control computer 550, the simulator can comprise any suitable component(s). In general, a “simulator” refers to the hardware and/or software components (e.g., a processor executing computer-readable instruction code) that allow a simulation to be executed on the surgeon bridge 100 to allow a user to practice using the user input devices of the surgeon bridge 100. As discussed in more detail below, the simulation can be of a simulated surgery and/or present exercises that are not of a simulated surgery but nonetheless teach the user specific psychomotor skills/memory that may be helpful in performing actual surgery using the robotic surgery system 150.

As mentioned above, user input signals are sent from the surgeon bridge controller 520 to the control tower switch 530 during actual surgery but are sent to the simulator (in this example, the simulator control computer 540) during simulation training. In this embodiment, the surgeon bridge mode switch 510 is used to direct this traffic. In general, the surgeon bridge mode switch 510 is a managed hub that creates communication channels between the various ports and can be used to switch between clinical and simulation modes. In one embodiment, the surgeon bridge mode switch 510 is a managed switch that creates a virtual local area network (VLAN) between different ports in the switch 510. (While VLANs are used in these examples, it should be noted that other types of channels can be used.) By being managed, the surgeon bridge mode switch 510 is configured to allow connections between some of the ports while restricting connections between other ports.

For example, as shown in FIG. 5A, when the robotic surgical system 150 is in simulation mode, the surgeon bridge mode switch 510 creates a VLAN 560 between the simulator control computer 540 and the surgeon bridge computer 520. As indicated by the “X” in this figure, the switch 510 does not create a VLAN communication channel between the simulator control computer 540 and the control tower switch 530 (other channels, such as a power management channel, can still be kept open). That is, in simulation mode, the switch 510 is configured to establish a VLAN (or other type of communication channel) between the surgeon bridge computer 520 and the simulator control computer 540, such that signals generated by the surgeon bridge computer 520 in response to user-manipulation of the plurality of user input devices are provided to the simulator control computer 540 for the simulation exercise but do not cause movement of robotic arms of the robotic surgical system 150. So, during simulation mode, the surgeon bridge computer 420 is disconnected from the control tower switch 530, thus providing a safety feature ensuring that any user input from the surgeon bridge computer 520 is only driving the simulation and not the actual surgical robot. Thus, this embodiment replicates the user experience of the real robot while ensuring that the simulation/training does not interact with the robot arms.

Similarly, for security and safety, it may be desired to isolate the simulator rendering computer 550 (e.g., a Windows-based personal computer (PC)) from the clinical components. This embodiment accomplishes this by creating another VLAN 570 between just the simulator rendering computer 550 and the simulator control computer 540.

As shown in FIG. 5B, when the user switches modes (e.g., by selecting a virtual button on the GUI to end the simulation), the robotic surgical system 150 causes the switch 510 to disable the VLAN 560 between the surgeon bridge computer 510 and the simulator control computer 540 and establish a VLAN 590 (or other type of communication channel) between the surgeon bridge computer 510 and a robotic arm controller of the robotic surgical system via the control tower computer 530, thus switching from simulation mode to clinical mode. In this mode, signals generated by the surgeon bridge computer in response to user-manipulation of the plurality of user input devices cause movement of robotic arms of the robotic surgical system 150.

As noted above, because the simulator in this embodiment is integrated in the surgeon bridge 100, switching between modes can be done with a simple user interface selection, which avoids the set-up and tear-down hassles associated with using a separate machine and its accompanying cabling. Further, since the simulator is integrated into the surgeon bridge 100, any surgeon bridge can serve as a stand-alone simulator. Further, simulation and training can be synched based on user logins and profiles, thus not tied with any particular simulator/surgeon bridge.

As mentioned above, the simulator can be used to provide exercise activities, procedure steps, and evaluation metrics in order to practice psychomotor skills on the same surgeon bridge 100 that will be used to perform actual surgery. The robotic surgical system 150 comprises an ergonomic layout on which a surgeon needs to perform specific psychomotor skills in order to be successful during a procedure. Thus, the simulation can help the surgeon learn the location of the controllers (buttons, pedals, etc.), their actions (depending on the system state), and the feedbacks from the system. The training simulator allows the surgeon to build safety, efficiency, and confidence, thus shortening the learning curve on the real robotic system.

Repetition is a very effective method to master psychomotor skills, and the simulator can provide core skills exercises for basic training that have the user perform simple tasks in a 3D simulated environment that is run several times. The following paragraphs describe a solution to take the level of granularity of the repetitions a step finer. In one embodiment, the exercise design has the user repeat atomic actions (e.g., a pedal press) or a sequence of actions (e.g., engagement) to build up psychomotor memory. Ultimately, associating the idea of a controller or action to the correct movement (e.g., of the user's foot) will become second nature. This is facilitated by user-profile-based ergonomics.

As mentioned above, one of the primary objectives of the training simulator is to reduce the length of the learning curve for new users on the robotic surgical system 150. Mastering psychomotor skills and memory is one of the factors coming into play at the beginning of the learning curve, and the training simulator can be used to increase the gradient of the learning curve. For example, in using an immersive display, one would have to move one's head back to be able to see the pedals. In using an open display, looking at the pedals might disengage the robot because of the eye tracker. Knowing both the location of the pedals and having the ability to move one's foot to the right spot becomes essential to increasing the efficiency and confidence of the user. Also, the simulator can be used after the initial training to perfect skills.

To keep the user engaged, the exercises provided by the simulator can be designed as a fun game that motivates the users to come back to get a better score and engage with the other simulation exercises outside of the training. For example, the simulator can present a memory game in which the user has to perform a list of actions and adds an action to be performed in repeat plays of the game. As another example, the simulator can present a scrolling/running list of actions to perform, similar to the Guitar Hero™ video game. Of course, these are merely examples, and other types of exercises can be used.

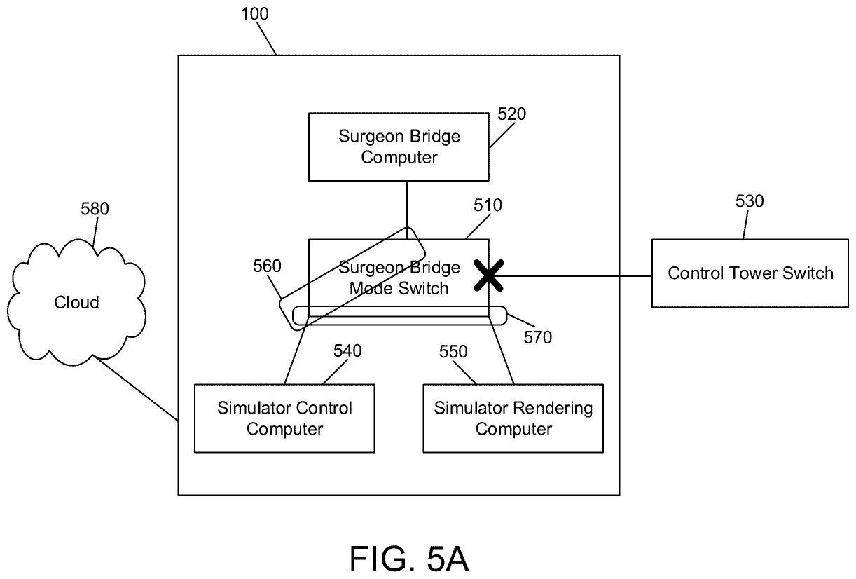

Turning again to the drawings, FIG. 6 is a flow chart 600 of a method of an embodiment for providing a training exercise on the simulator. As shown in FIG. 6, first, the GUI prompts the user to perform an action on the surgeon bridge 120 (e.g., either by the name of the controller or by the action associated with it) (act 610). Next, the user performs the action as fast as possible (act 620). The GUI then shows if the action has been performed correctly or not (act 630). This loop can be repeated a number of times.

Several variations of this workflow are possible depending on the level of difficulty desired. For example, there can be a “no-timeout mode” in which the user can take however long they need to perform the action, a “timeout mode” in which the user has a limited time to perform the action (if the action times out, the loop can go to the next action), a “timeout-length-decreases mode” in which the user has less and less time to perform the action, and an “infinite-loop mode” in which the time-out length decreases and loops until the user fails an action. Also, to increase the level of difficulty, the action prompted can be an atomic action (e.g., pedal press) followed by an “usual” action if identified from an usual clinical use (e.g., clutching after moving the camera) or a sequence of actions (e.g., engagement).

While the exercise can be performed in a 3D-simulated environment, other options are possible. For example, the GUI can display a picture of the surgeon bridge 100 to give hints to the user or can display a stack of the actions for the user to anticipate the next actions. In other embodiments, no visual clues are given.

As mentioned above, the user's performance can be evaluated based on criteria. Such criteria can include, but is not limited to, the number of correctly performed actions, the number of wrong actions, the number of time-outs, the reaction time per action, and comparative performance with fellow trainees or benchmarks.

The list/order of actions to be performed in the simulation can be totally random or semi-random (to cover the whole surgeon bridge 100), or can use simple data analytics to identify actions that the user has trouble performing (e.g., those that have a longer reaction time or resulted in a wrong action) and insist on these the next time the user runs the exercise.

### Training Exercise to Understand the Workspace of an Ungrounded Input Device

Another example of a simulation is a training exercise to understand the workspace of an ungrounded input device. The simulator for this type of training can be integrated or non-integrated in the surgeon bridge 100. In order for a surgeon to be successful in controlling the end-effector instruments of the robotic arms during robotic surgery, he needs to learn how to use the input devices within the constraints of the specific robotic platform. As users transition between robotic platforms, it becomes increasingly important that there are ways for a surgeon to quickly and easily become familiar with any system-specific constraints that impact their ability to control instruments.

In one embodiment, some of the user input devices of the robotic surgical system 150 are ungrounded input devices (UIDs) held in the user's hands to move the surgical instruments. These are “ungrounded” in the sense that there are no mechanical constraints on the user moving the devices to any location. In contrast, “grounded” input devices may be on arms that have a hard stop to prevent movement of the devices beyond the mechanical limitations set by the arms. Ungrounded input devices can be tracked in space with an electro-magnetic (EM) tracker. UID movements are then converted to instrument motion through robotics control (RC) algorithms. The accuracy of EM tracking decreases if the UIDs are too far from or too close to the tracker. The user needs to keep his hands in the usable workspace so that the system can track the UIDs accurately enough for teleoperation. The size and shape of this workspace is typically characterized by the manufacturer of the tracker and is referred herein as the “EM (or UID) workspace.”

In one embodiment, the boundaries of this workspace are not visible, and the system 150 cannot physically prevent the user from moving the input devices outside of this workspace. This presents a system-specific constraint that a surgeon user must become familiar with in order to effectively control the robot. The size of this workspace does not allow the user to move the instruments in their full range of reach and access without having to reposition their hands, depending on the motion scaling. As used herein, “tool reach and access” refers to the space defined by the arm joints limits, and “tool workspace” refers to the space defined by the EM workspace.

In one embodiment, a “clutching” operation is used to reposition hands in the workspace without moving the robotic arms. For example, in one implementation, a user presses a clutch pedal, which allows the user to move the UIDs without moving the robotic arms. In this way, the user can reposition his hands without moving the robotic arms. Once the user's hands are in a desired location, the user can release the clutch pedal, after which movement of UIDs again causes the robotic arms to move.

The following embodiments can be used to allow the user to quickly become familiar with the system constraints, conceptualize the EM workspace boundaries, understand the impact of hand positioning on instrument control, adjust tool workspace by clutching, and understand the impact of camera motion on tool workspace (if camera motion is controlled with hand movement).

In one embodiment, if the user moves one or both of the input devices outside of the EM workspace boundaries, the robotic surgical system 150 will automatically disengage the user from active teleoperation. The user will then have to reposition his hands inside the EM workspace and re-engage. This could disrupt or slow down the surgical procedure. The concept of clutching and repositioning hands to be able to move the instruments farther is part of the teleoperation of the surgical robot of this embodiment. New surgeons can struggle with understanding how hand motion translates to instrument motion and how hand positioning impacts the tool workspace. Similarly, new users can struggle to understand the impact of camera motion on tool workspace when endoscope motion is controlled with the hand-held input devices. The motion of the camera is the opposite of the hands motion (move back to zoom in); thus, the user's hands match the relative movement of the instruments with reference to the camera view, automatically redefining the tool workspace (indirectly achieving the same as clutching).

To address the above problems, the simulator can provide a simulation exercise to allow the surgeon to learn how to control the user input devices within the available workspace and ergonomics of the system. The exercise can be a 3D scene whose camera view is the endoscope. The scene can include two instruments (e.g., one controlled with the left UID, and the other controlled with the right UID). The 3D scene can either be empty (with some background for scale) or have objects with which to interact.

For simplification, the drawings presented herein are 2D, and the tool workspace is a rectangle. However, it should be noted that, in operation, the simulator can present the tool workspace as a parallelepiped. Also to simplify the drawings, the motion scaling is 1:1 (i.e., the tool workspace is as big as the EM workspace). For further simplification, some of the drawings will show only one UID and instrument.

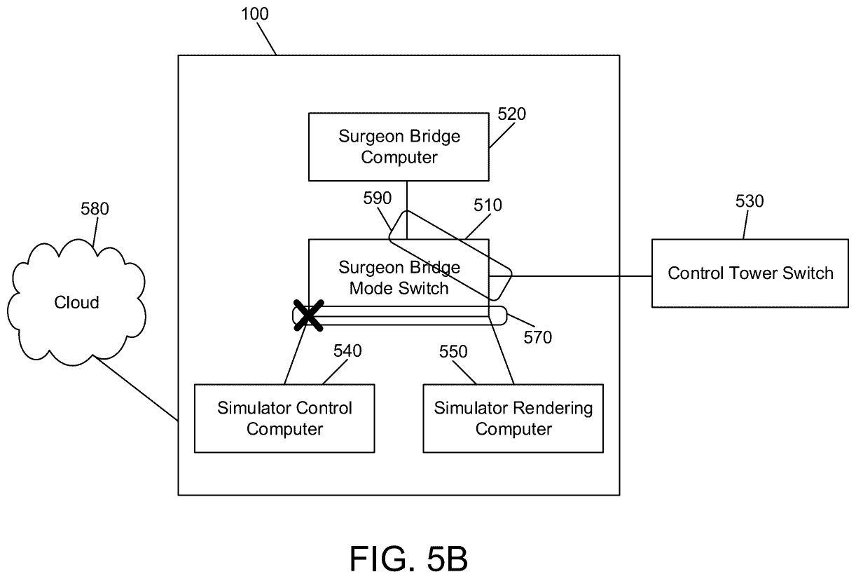

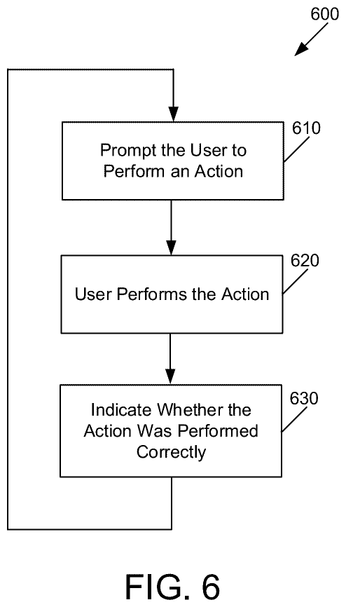

Returning to the drawings, FIG. 7 is an illustration of a simulation exercise of an embodiment that is displayed on the display device of the surgeon bridge 100. As shown in FIG. 7, this simulation shows the left UID and its EM workspace, as well as the left tool and its reach. As shown in this illustration, a given position of a UID inside the EM workspace determines the workspace of the corresponding instrument. If the user's hand is “aligned” with the tool, the tool workspace is “mapped” to the EM workspace. This is shown in FIG. 7 with the position of the left UID in the EM workspace being the same or about the same as the position of the left tool in the left tool reach box. In active teleoperation, when moving the left UID, the tool follows, and the tool workspace does not move. This is shown in FIG. 8, which shows that when the left UID is near the right edge of the EM workspace, the left tool is near the right edge of the tool reach.

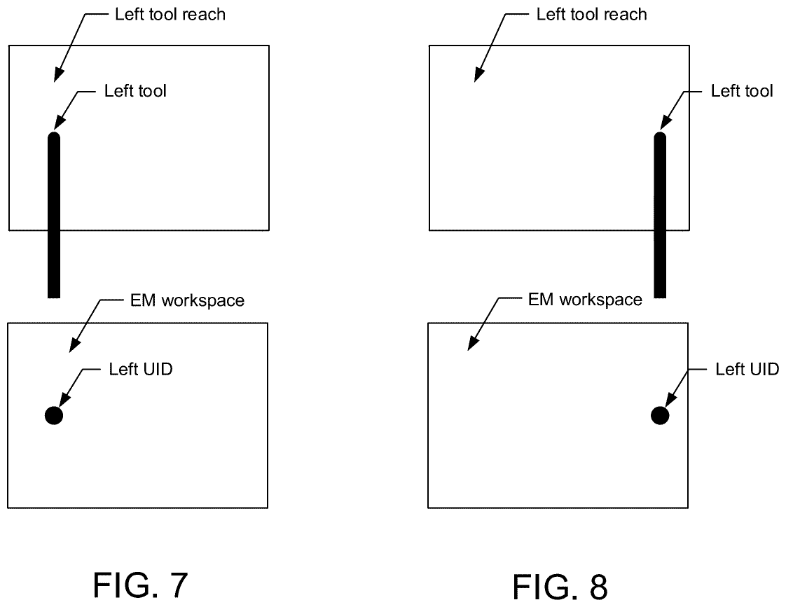

If the user desires to move the tool further to the right, the user will encounter a problem, as he is nearing the right-most edge of the EM workspace. In this situation, the user would press the clutch pedal and move his hand to the left. With the clutch pedal depressed, moving the left UID does not move the tool, but it does move the workspace of the tool. This is shown in FIG. 9, which shows that, through this repositioning operation, the left UID is now in the lower left-hand corner of the EM workspace, and the left tool is in the lower left-hand corner of the tool reach box. This allows the user to move the left UID and left tool further to the right.

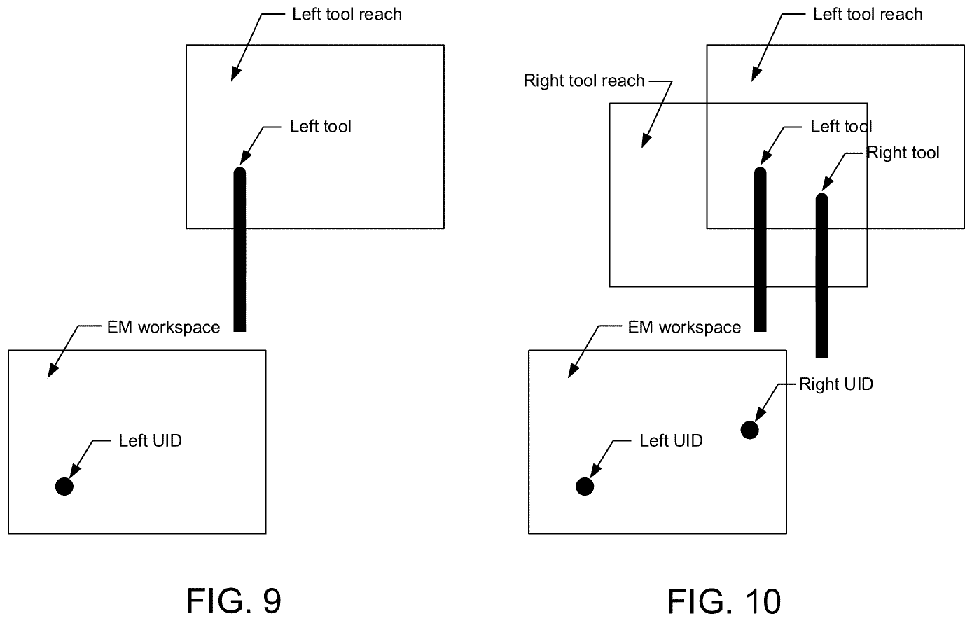

In this embodiment, when two instruments are used, the relationship between the UID workspace and the tool workspace is independent for each tool. This is shown in FIG. 10, which shows that the left and right UIDs can be moved or clutched independently.

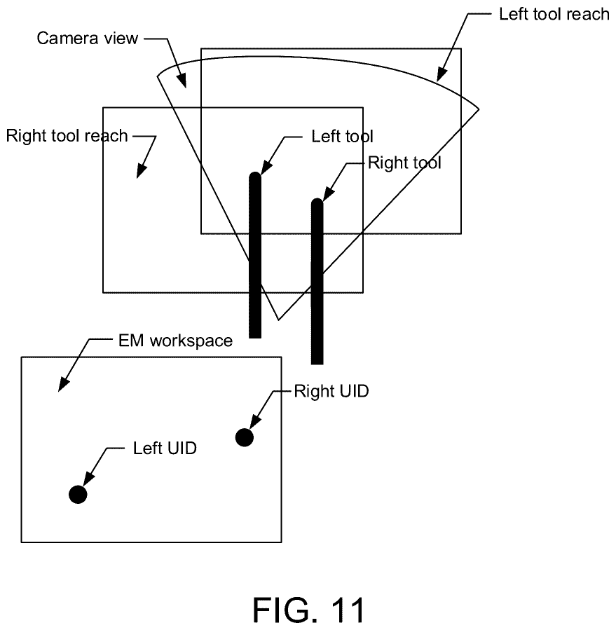

In addition to the tools, the UID can be used to move the endoscope camera to change the field of view. In one embodiment, moving the camera (e.g., done by moving the UID with the one of the pedals pressed) clutches both instruments at the same time. So, in this embodiment, moving the camera has the same effect on the tool workspace as clutching and repositioning the UIDs but also changes the point of view of the surgeon. Since the camera moves in the opposite direction of the UID, the tool workspace moves with the camera, unless the hands are moved apart or closer to each other, which does not have any action on the camera but will change the tool workspace. This is shown in FIG. 11.

There are many alternatives that can be used with these embodiments. These include, but are not limited to, GUI indication of EM workspace boundaries, 3D visualization of EM workspace and input devices on screen, augmented reality (AR) glasses to visualize EM workspace boundaries around the user's hands, and a clear plastic training box that attaches to the tracker to physically embody/materialize the EM workspace boundaries.

Other Illustrative Embodiments include the following. Illustrative Embodiments for one type of claim (e.g., system, method, computer program, or computer readable storage medium) may be provided in other types (e.g., system as a method). Illustrative Embodiments for one set (e.g., Illustrative Embodiments 1-9) may be used in other sets.

Illustrative Embodiment 1. A robotic surgical system user console comprising: a plurality of user input devices; a processor configured to generate signals in response to user-manipulation of the plurality of user input devices; a simulator configured to provide a simulation exercise; and a switch configured to operate in a clinical mode or a simulation mode; wherein, in the clinical mode, the switch is configured to establish a connection between the processor and a robotic arm controller of the robotic surgical system, such that signals generated by the processor in response to user-manipulation of the plurality of user input devices cause movement of robotic arms of the robotic surgical system; and wherein, in the simulation mode, the switch is configured to establish a connection between the processor and the simulator, such that signals generated by the processor in response to user-manipulation of the plurality of user input devices are provided to the simulator for the simulation exercise but do not cause movement of robotic arms of the robotic surgical system.

Illustrative Embodiment 2. The robotic surgical system user console of Illustrative Embodiment 1, wherein the simulator is integrated in the robotic surgical system user console.

Illustrative Embodiment 3. The robotic surgical system user console of any of Illustrative Embodiments 1-2, wherein the simulation exercise teaches psychomotor skills required in surgical procedures.

Illustrative Embodiment 4. The robotic surgical system user console of any of Illustrative Embodiments 1-3, wherein the simulation exercise has a user repeat atomic actions or a sequence of actions using the plurality of user input devices to build psychomotor memory.

Illustrative Embodiment 5. The robotic surgical system user console of any of Illustrative Embodiments 1-4, wherein in the clinical mode, the switch is configured to establish a virtual local area network between the processor and the robotic arm controller, and wherein, in the simulation mode, the switch is configured to establish a virtual local area network between the processor and the simulator.

Illustrative Embodiment 6. The robotic surgical system user console of any of Illustrative Embodiments 1-5, wherein the simulator comprises a simulator control computer and a simulator rendering computer.

Illustrative Embodiment 7. The robotic surgical system user console of Illustrative Embodiment 6, wherein the switch is further configured to establish a virtual local area network between the simulator control computer and the simulator rendering computer.

Illustrative Embodiment 8. The robotic surgical system user console of any of Illustrative Embodiments 1-7, wherein the simulator is configured to communicate with a network cloud.

Illustrative Embodiment 9. The robotic surgical system user console of any of Illustrative Embodiments 1-8, wherein the switch is configured to establish the connection between the processor and the robotic arm controller via a control tower switch.

Illustrative Embodiment 10. A method for performing a robotic surgical system simulation, the method comprising: performing the following in a surgeon bridge of a robotic surgical system comprising a plurality of user input devices; a simulator configured to provide a simulation exercise; and a switch configured to operate in a clinical mode or a simulation mode: in response to receiving a command to operate in the clinical mode, using the switch to establish a connection with a robotic arm controller of the robotic surgical system, such that signals generated in response to user-manipulation of the plurality of user input devices cause movement of robotic arms of the robotic surgical system; and in response to receiving a command to operate in the simulation mode, using the switch to establish a connection with the simulator, such that signals generated in response to user-manipulation of the plurality of user input devices are provided to the simulator for the simulation exercise but do not cause movement of robotic arms of the robotic surgical system.

Illustrative Embodiment 11. The method of Illustrative Embodiment 10, wherein the simulator is integrated in the robotic surgical system user console.

Illustrative Embodiment 12. The method of any of Illustrative Embodiments 10-11, wherein the simulation exercise teaches psychomotor skills required in surgical procedures.

Illustrative Embodiment 13. The method of any of Illustrative Embodiments 10-12, wherein the simulation exercise has a user repeat atomic actions or a sequence of actions using the plurality of user input devices to build psychomotor memory.

Illustrative Embodiment 14. The method of any of Illustrative Embodiments 10-13, wherein the connection with the robotic arm controller and the connection with the simulator are made using different virtual local area networks.

Illustrative Embodiment 15. A robotic surgical system user console comprising: a plurality of user input devices; means for allowing user-manipulation of the plurality of user input devices to cause movement of robotic arms of the robotic surgical system when in a clinical mode; and means for preventing user-manipulation of the plurality of user input devices from causing movement of robotic arms of the robotic surgical system when in a simulation mode.

Illustrative Embodiment 16. The robotic surgical system user console of Illustrative Embodiment 15, wherein the means for allowing establishes a virtual local area network with a robotic arm controller.

Illustrative Embodiment 17. The robotic surgical system user console of any of Illustrative Embodiments 15-16, wherein the means for preventing establishes a virtual local area network with a simulator and not with a robotic arm controller.

Illustrative Embodiment 18. The robotic surgical system user console of any of Illustrative Embodiments 15-17, further comprising means for providing a simulation exercise that teaches psychomotor skills required in surgical procedures.

Illustrative Embodiment 19. The robotic surgical system user console of any of Illustrative Embodiments 15-18, further comprising means for providing a simulation exercise that has a user repeat atomic actions or a sequence of actions using the plurality of user input devices to build psychomotor memory.

Illustrative Embodiment 20. The robotic surgical system user console of any of Illustrative Embodiments 15-19, further comprising: a simulator comprising a simulator control computer and a simulator rendering computer; and means for establish a virtual local area network between the simulator control computer and the simulator rendering computer.

Illustrative Embodiment 21. A robotic surgical system user console comprising: a user input device; an electromagnetic tracker configured to track a position of the user input device; a display device; and a processor configured to provide a simulation exercise that displays on the display device: a representation of an electromagnetic workspace; an indication of a location of the user input device in the electromagnetic workspace; a representation of a tool workspace; and an indication of a location of a tool in the tool workspace; wherein movement of the user input device results in movement of the indication of the location of the user input device in the representation of the electromagnetic workspace and the indication of the location of the tool in the representation of the tool workspace.

Illustrative Embodiment 22. The robotic surgical system user console of Illustrative Embodiment 21, further comprising a clutch pedal, wherein in response to the clutch pedal being depressed, movement of the user input device results in movement of the indication of the location of the user input device in the representation of the electromagnetic workspace but does not result in movement of the indication of the location of the tool in the representation of the tool workspace.

Illustrative Embodiment 23. The robotic surgical system user console of Illustrative Embodiment 22, wherein in response to the clutch pedal being released, movement of the user input device results in movement of the indication of the location of the user input device in the representation of the electromagnetic workspace and in movement of the indication of the location of the tool in the representation of the tool workspace.

Illustrative Embodiment 24. The robotic surgical system user console of any of Illustrative Embodiments 21-23, wherein the representation of the electromagnetic workspace and the representation of the tool workspace are displayed in three dimensions.

Illustrative Embodiment 25. The robotic surgical system user console of any of Illustrative Embodiments 21-24, further comprising a second user input device, wherein a relationship between the electromagnetic workspace and the tool workspace of the user input device is independent of a relationship between an electromagnetic workspace and a tool workspace of a second user input device.

Illustrative Embodiment 26. The robotic surgical system user console of any of Illustrative Embodiments 21-25, wherein moving a camera of the robotic surgical system to change a field of view also causes a change in movement of the indication of the location of the user input device in the representation of the electromagnetic workspace.

Illustrative Embodiment 27. The robotic surgical system user console of any of Illustrative Embodiments 21-26, wherein the user input device is ungrounded.

Illustrative Embodiment 28. A method for performing a robotic surgical system simulation, the method comprising: performing the following in a surgeon bridge of a robotic surgical system comprising a user input device; an electromagnetic tracker configured to track a position of the user input device; and a display: displaying a simulation on the display device, wherein the simulation displays a representation of an electromagnetic workspace, an indication of a location of the user input device in the representation of the electromagnetic workspace, a representation of a tool workspace, and an indication of a location of a tool in the representation of the tool workspace; and in response to movement of the user input device, moving the indication of the location of the user input device in the representation of the electromagnetic workspace and moving the indication of the location of the tool in the representation of the tool workspace.

Illustrative Embodiment 29. The method of Illustrative Embodiment 28, further comprising in response to the clutch pedal being depressed and movement of the user input device, moving the indication of the location of the user input device in the representation of the electromagnetic workspace but not the indication of the location of the tool in the representation of the tool workspace.

Illustrative Embodiment 30. The method of Illustrative Embodiment 29, wherein in response to the clutch pedal being released and movement of the user input device, moving the indication of the location of the user input device in the representation of the electromagnetic workspace and the indication of the location of the tool in the representation of the tool workspace.

Illustrative Embodiment 31. The method of any of Illustrative Embodiments 28-30, wherein the representation of the electromagnetic workspace and the representation of the tool workspace are displayed in three dimensions.

Illustrative Embodiment 32. The method of any of Illustrative Embodiments 28-31, wherein a relationship between the electromagnetic workspace and the tool workspace of the user input device is independent of a relationship between an electromagnetic workspace and a tool workspace of a second user input device.

Illustrative Embodiment 33. The method of any of Illustrative Embodiments 28-32, further comprising in response to movement of a camera of the robotic surgical system to change a field of view, moving the indication of the location of the user input device in the representation of the electromagnetic workspace.

Illustrative Embodiment 34. The method of any of Illustrative Embodiments 28-33, wherein the user input device is ungrounded.

Illustrative Embodiment 35. A robotic surgical system user console comprising: a display device; a user input device; an electromagnetic tracker configured to track a position of the user input device; and means for providing a simulation exercise that displays on the display device: a representation of an electromagnetic workspace; an indication of a location of the user input device in the representation of the electromagnetic workspace; a representation of a tool workspace; and an indication of a location of a tool in the representation of the tool workspace; wherein movement of the user input device results in movement of the indication of the location of the user input device in the representation of the electromagnetic workspace and in movement of the indication of the location of the tool in the representation of the tool workspace.

Illustrative Embodiment 36. The robotic surgical system user console of Illustrative Embodiment 35, further comprising a clutch pedal, wherein in response to the clutch pedal being depressed, movement of the user input device results in movement of the indication of the location of the user input device in the representation of the electromagnetic workspace but does not result in movement of the indication of the location of the tool in the representation of the tool workspace.

Illustrative Embodiment 37. The robotic surgical system user console of any of Illustrative Embodiments 35-36, wherein in response to the clutch pedal being released, movement of the user input device results in movement of the indication of the location of the user input device in the representation of the electromagnetic workspace and in movement of the indication of the location of the tool in the representation of the tool workspace.

Illustrative Embodiment 38. The robotic surgical system user console of any of Illustrative Embodiments 35-37, wherein the representation of the electromagnetic workspace and the representation of the tool workspace are displayed in three dimensions.

Illustrative Embodiment 39. The robotic surgical system user console of any of Illustrative Embodiments 35-38, further comprising a second user input device, wherein a relationship between the electromagnetic workspace and the tool workspace of the user input device is independent of a relationship between an electromagnetic workspace and a tool workspace of a second user input device.

Illustrative Embodiment 40. The robotic surgical system user console of any of Illustrative Embodiments 35-39, wherein moving a camera of the robotic surgical system to change a field of view also causes a change in movement of the indication of the location of the user input device in the representation of the electromagnetic workspace.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

What is claimed is:

1. A robotic surgical system user console comprising:
a plurality of user input devices;
a processor configured to generate signals in response to user-manipulation of the plurality of user input devices;
a simulator configured to provide a simulation exercise; and
a switch configured to operate in a clinical mode or a simulation mode;
wherein, in the clinical mode, the switch is configured to establish a connection between the processor and a robotic arm controller of the robotic surgical system, such that signals generated by the processor in response to user-manipulation of the plurality of user input devices cause movement of robotic arms of the robotic surgical system; and
wherein, in the simulation mode, the switch is configured to establish a connection between the processor and the simulator, such that signals generated by the processor in response to user-manipulation of the plurality of user input devices are provided to the simulator for the simulation exercise but do not cause movement of robotic arms of the robotic surgical system.

2. The robotic surgical system user console of claim 1, wherein the simulator is integrated in the robotic surgical system user console.

3. The robotic surgical system user console of claim 1, wherein the simulation exercise teaches psychomotor skills required in surgical procedures.

4. The robotic surgical system user console of claim 1, wherein the simulation exercise has a user repeat atomic actions or a sequence of actions using the plurality of user input devices to build psychomotor memory.

5. The robotic surgical system user console of claim 1, wherein in the clinical mode, the switch is configured to establish a virtual local area network between the processor and the robotic arm controller, and wherein, in the simulation mode, the switch is configured to establish a virtual local area network between the processor and the simulator.

6. The robotic surgical system user console of claim 1, wherein the simulator comprises a simulator control computer and a simulator rendering computer.

7. The robotic surgical system user console of claim 6, wherein the switch is further configured to establish a virtual local area network between the simulator control computer and the simulator rendering computer.

8. The robotic surgical system user console of claim 1, wherein the simulator is configured to communicate with a network cloud.

9. The robotic surgical system user console of claim 1, wherein the switch is configured to establish the connection between the processor and the robotic arm controller via a control tower switch.

10. A method for performing a robotic surgical system simulation, the method comprising:
performing the following in a surgeon bridge of a robotic surgical system comprising a plurality of user input devices; a simulator configured to provide a simulation exercise; and a switch configured to operate in a clinical mode or a simulation mode:
in response to receiving a command to operate in the clinical mode, using the switch to establish a connection with a robotic arm controller of the robotic surgical system, such that signals generated in response to user-manipulation of the plurality of user input devices cause movement of robotic arms of the robotic surgical system; and
in response to receiving a command to operate in the simulation mode, using the switch to establish a connection with the simulator, such that signals generated in response to user-manipulation of the plurality of user input devices are provided to the simulator for the simulation exercise but do not cause movement of robotic arms of the robotic surgical system.

11. The method of claim 10, wherein the simulator is integrated in the robotic surgical system user console.

12. The method of claim 10, wherein the simulation exercise teaches psychomotor skills required in surgical procedures.

13. The method of claim 10, wherein the simulation exercise has a user repeat atomic actions or a sequence of actions using the plurality of user input devices to build psychomotor memory.

14. The method of claim 10, wherein the connection with the robotic arm controller and the connection with the simulator are made using different virtual local area networks.

15. A robotic surgical system user console comprising:
a plurality of user input devices;
means for allowing user-manipulation of the plurality of user input devices to cause movement of robotic arms of the robotic surgical system when in a clinical mode; and
means for preventing user-manipulation of the plurality of user input devices from causing movement of robotic arms of the robotic surgical system when in a simulation mode.

16. The robotic surgical system user console of claim 15, wherein the means for allowing establishes a virtual local area network with a robotic arm controller.

17. The robotic surgical system user console of claim 15, wherein the means for preventing establishes a virtual local area network with a simulator and not with a robotic arm controller.

18. The robotic surgical system user console of claim 15, further comprising means for providing a simulation exercise that teaches psychomotor skills required in surgical procedures.

19. The robotic surgical system user console of claim 15, further comprising means for providing a simulation exercise that has a user repeat atomic actions or a sequence of actions using the plurality of user input devices to build psychomotor memory.

20. The robotic surgical system user console of claim 15, further comprising:
a simulator comprising a simulator control computer and a simulator rendering computer; and
means for establish a virtual local area network between the simulator control computer and the simulator rendering computer.
